(19) United States
(12) Patent Application Publication
(10) Pub. No.: US 2008/0275393 A1
(43) Pub. Date: Nov. 6, 2008

(54) Isolation thrombectomy catheter system

(76) Inventors: Michael J. Bonnette, Minneapolis, MN (US); Hieu V. Le, Brooklyn Park, MN (US)

Correspondence Address: GREGORY L BRADLEY;MEDRAD INC, ONE MEDRAD DRIVE, INDIANOLA, PA, 15051, US

(21) Appl. No.: 10/925,113

(22) Filed: Aug. 24, 2004

(51) Int. Cl. A61M 1/00; A61M 25/10

(52) U.S. Cl. 604/102.01; 604/151

(57) ABSTRACT

An isolation thrombectomy catheter system where thrombus, clotted material, or the like is isolated by distally and proximally placed inflated occlusive balloons sealing a portion of the vasculature for subsequent removal of thrombus by cross stream jets. An isolation catheter accommodates either a jet assembly for ablation of thrombus and for drug or medication delivery, or accommodates a drug delivery device for introduction of lysing fluids, drugs or medications to a thrombus site. Alternating of lysing fluid delivery and ablation can be incorporated for a combined thrombectomy effort.

### CROSS REFERENCES TO RELATED APPLICATIONS

This patent application is related to patent application Ser. No. 09/888,455 entitled “Single Operator Exchange Fluid Jet Thrombectomy Device” filed Jun. 25, 2001, to be issued; and to patent application Ser. No. 10/455,096 entitled “Thrombectomy Catheter Device Having a Self-Sealing Hemostasis Valve” filed Jun. 5, 2003.

### BACKGROUND OF THE INVENTION

1. Field of the Invention

The present invention is for a catheter system, and more specifically, an isolation thrombectomy catheter system where thrombus, clotted material, and the like is isolated by distally and proximally placed inflated occlusive balloons and subsequently removed by jet streams, or lysed and removed, from arteries, veins, and the like.

2. Description of the Prior Art

Prior art thrombectomy devices have been utilized to loosen and remove deposited thrombotic material generally characterized as lesions, clots, or other material deposited on or clinging to the walls of arteries, vessels, or veins, which are often referred to collectively as the vasculature. Such prior art devices incorporate a catheter system incorporated with the use of a guidewire which places the distal end of the catheter into close proximity with the thrombotic material followed by positioning the distal end of a high pressure saline delivery tube at the distal end of the catheter and then causing high pressure fluid jets of saline to be directed directly at or through an orifice arrangement to impingingly contact the thrombotic material to cause such material to break up and be dislodged from the walls of the vasculature. Often, the dislodged thrombotic material is entrained into a recirculating flow for further maceration and then evacuated as effluence through the lumen of the catheter. Prior art devices, while providing for general evacuation of loosened and broken-up thrombotic material, have not provided for complete evacuation of broken-up thrombotic material. Consequently, broken-up thrombotic material may reform elsewhere in the vasculature, especially thrombotic material consisting of coagulated blood. Complete removal of broken-up thrombotic material is highly desirable, and failure to remove such will increase the possibility of embolization by non-evacuated coagulated blood distally in the vasculature.

Some prior art devices, while offering the capability of removing thrombotic material, clots, lesions, and the like, proved difficult or nearly impossible to navigate and place within tortuous paths within the vasculature due to the inflexibility of the distal end of the thrombectomy device. Often, prior art devices do not adequately address centering of a thrombectomy device within the vasculature, whereby high pressure fluid jets, being off center with respect to the vasculature walls, come into dangerously close proximity with the vasculature walls, thereby increasing the chances of breaking through or otherwise damaging the vasculature walls.

Other methods of removal or dissolution of clots is by the delivery of a drug by a catheter to the site of a blood clot to dissolve the clot. The delivery of the drug to the thrombus site may be incorporated by itself or by alternating the delivery of drugs and delivery of high pressure fluid jets of saline to rid a vein or vessel of thrombus. While delivery of a drug to the clot site may cause the clot to be partially softened and/or partially dissolved, the interacting effectiveness of the drug with the clot may be diluted and hampered by carrying away of the drug by blood flow transiting the clot site. If a clot is not fully dissolved, then additional applications of drugs can be required to complete the dissolving process, thereby adding an excessive amount of drugs to the overall vasculature system, the addition of which could be detrimental. In light of the shortcomings of the prior art devices, it is desirable to be able to completely isolate a thrombectomy site in order to prevent carriage of all types of broken-up thrombus from recirculation in the vascular system. Another desirable benefit can include the ability to also controllably and isolatingly deliver clot dissolving drugs to an isolated thrombosis site.

### SUMMARY OF THE INVENTION

The general purpose of the present invention is to provide an isolation thrombectomy catheter system. The system is utilized to isolate a blood clot or other origin thrombotic lesions located on the inner walls of blood veins, arteries, and the like, whereby a thrombectomy procedure ensues. Removal of thrombus can be facilitated by impingement of cross stream jets with the thrombus followed by maceration and exhausting of thrombus. Additionally, the isolation thrombectomy catheter system is also valuable in delivery of drugs to an isolated site of the vasculature to come in contact with and to soften or weaken the thrombus deposit followed by impingement of cross stream jets with the thrombus followed by maceration and exhausting of thrombus.

According to one or more embodiments of the present invention, there is provided an isolation thrombectomy catheter system including a centrally located manifold to which a plurality of components are connected and through which a plurality of components pass, where such components include an isolation catheter and treatment apparatus which maintain substantially a coaxial relationship. The isolation catheter generally includes a flexible catheter tube having a plurality of features distributed along the length thereof including a tip which is tapered and flexible, a distal occlusive balloon which is flexible, a proximal occlusive balloon which is flexible, and a plurality of window orifices located between the distal occlusive balloon and the proximal occlusive balloon. The inclusion of window orifices between the distal occlusive balloon, which is flexible, and the proximal occlusive balloon, which is flexible, provides for overall flexibility of the distal portion of the catheter tube, thereby enabling navigation through tortuous paths of the vasculature. The proximal end of the isolation catheter terminates at and communicates with the interior of the manifold. The catheter tube includes an offset minimum profile inflation lumen communicating with the proximally and distally located occlusive balloons and also contains a larger central lumen. An inflation port extends from the manifold to accommodate an inflation syringe both of which communicate with the distal and proximal occlusive balloons through the minimum profile inflation lumen. The treatment apparatus can comprise a jet assembly which includes a flexible partial length jet catheter having at least a plurality of distally located inflow and outflow orifices, a high pressure tube, a fluid jet emanator, and a manual actuator, and along with the jet assembly, a high pressure pump and a fluid (e.g., saline) supply, such components being mutually connected; or the treatment apparatus can be a drug delivery device composed of a drug delivery catheter having drug dispensing ports and other structure, along with a drug pump and a drug supply, such components being mutually connected. Parts and portions of the jet assembly align within a hemostatic sealing arrangement at the proximal end of the manifold, within the manifold, and within the isolation catheter. In operation, the isolation catheter is positioned in close proximity to a clot, a lesion or thrombus area of the vasculature, such that the uninflated distally and proximally located occlusive balloons are positioned for subsequent inflation, thereby opposingly sealing against the vein or blood vessel to include the clot, thrombus or lesion in an isolated environment. The high pressure tube, the fluid jet emanator, and the partial length jet catheter of the jet assembly are advanced along the interior of the isolation catheter to position the fluid jet emanator in alignment between and for to and fro positional actuation between the inflated distal and proximal occlusive balloons of the isolation catheter. Such a placement and actuation along and within the isolated environment places the fluid jet emanator along and in close proximity to the plurality of window orifices at the distal end of the isolation catheter where fluid jets streaming forth from the fluid jet emanator create cross stream flows between the outflow and the inflow orifices. Such cross stream flows between the outflow and the inflow orifices align within the various plurality of window orifices to erode away the thrombus, clots or other foreign material to macerate the thrombus, clots, or other foreign material and to carry away macerated thrombus or thrombotic-like deposits along the central lumen of the isolation catheter for disposal. A portion of the partial length jet catheter can be extended beyond the distal end of the isolation catheter to engage in breakup, maceration and evacuation of the undesirable materials distal to the isolation catheter, while maintaining a double seal proximal to the undesirable material deposit, thereby providing another use of the invention. Even another use is available using elements of the invention. Specifically, a treatment apparatus in the form of a separate drug delivery device fashioned according to the general shape of the partial length jet catheter can be provided for introduction through the manifold and the isolation catheter to deliver drugs, i.e., medications, by a drug pump from a drug supply to the window orifices at the distal end of the isolation catheter.

One significant aspect and feature of the present invention is the provision of an isolation thrombectomy catheter system having treatment apparatus of different types.

Another significant aspect and feature of the present invention is an isolation thrombectomy catheter system having an inflatable and deflatable distal occlusive balloon and an inflatable and deflatable proximal occlusive balloon at the distal end of a catheter tube of an isolation catheter.

Still another significant aspect and feature of the present invention is the provision and use of an inflatable and deflatable distal occlusive balloon and an inflatable and deflatable proximal occlusive balloon at the distal end of a catheter tube of an isolation catheter to seal off and isolate a region of the vasculature containing thrombus, whereby a thrombectomy procedure may be accomplished without the introduction of thrombus or like material into the bloodstream, thus greatly diminishing the possibility of embolization.

Yet another significant aspect and feature of the present invention is the provision and use of multiple window orifices at the distal end of a catheter tube of an isolation catheter, where such window orifices are located between an inflatable and deflatable distal occlusive balloon and an inflatable and deflatable proximal occlusive balloon at the distal end of the catheter tube of the isolation catheter.

A further significant aspect and feature of the present invention is the provision and use of a treatment apparatus including a distally located partial length jet catheter having a plurality of inflow orifices and a plurality of outflow orifices located at the distal end.

A still further significant aspect and feature of the present invention is a fluid jet emanator which can be aligned for use with a plurality of inflow orifices and a plurality of outflow orifices located at the distal end of a partial length jet catheter where cross stream jets pass through and operate in cooperation with window orifices located between an inflatable and deflatable distal occlusive balloon and an inflatable and deflatable proximal occlusive balloon at the distal end of a catheter tube of an isolation catheter to impinge, ablate, macerate, and remove thrombus.

A still further significant aspect and feature of the present invention is a longitudinally positionable and rotationally positionable relationship between a jet assembly and an isolation catheter, whereby a partial length jet catheter of the jet assembly can be positioned along the distal portion of the isolation catheter to align to various window orifices, thereby providing cross stream jet access to thrombus-laden areas of the vasculature, especially those areas which have been isolated by the occlusive balloons.

Yet another significant aspect and feature of the present invention is the centering of the distal portions of the isolation thrombectomy catheter system in a body vessel or cavity by the inflation of the distal and proximal occlusive balloons, thereby preventing impingement and blocking of the window orifices, such as by the occurrence of contact with the side of the body vessel or cavity.

Yet another significant aspect and feature of the present invention is the centering of the distal portions of the isolation thrombectomy catheter system in a body vessel or cavity by the inflation of the distal and proximal occlusive balloons, thereby providing a suitable space between the fluid jet and/or the cross stream jet flow and the body vessel or cavity.

Still another significant aspect and feature of the present invention is the ability of the distal portion of the jet assembly, i.e., the partial length jet catheter, to extend partially through the distal end of the isolation catheter so as to be utilizable as a thrombectomy catheter while being afforded a proximally located double seal as provided by the proximal and distal occlusive balloons.

Another significant aspect and feature of the present invention is the provision and use of a device for delivering drugs, i.e., medications, to the thrombus site to assist in cooperation with cross stream jets to rid the site of thrombus deposits.

Yet another significant aspect and feature of the present invention is the ability to utilize the isolation catheter with a drug delivery device to deliver thrombus softening drugs, i.e., medications, to an isolated thrombus site, thus keeping the drugs isolated for best use and also keeping the drugs from being unwantingly delivered or dispensed to other regions of the vasculature.

Still another significant aspect and feature of the present invention is the provision and use of an isolation catheter and treatment apparatus to deliver pressurized ablative medium to a thrombus site for ablation and evacuation of thrombus and the like.

Still another significant aspect and feature of the present invention is the provision and use of an isolation catheter and a drug delivery device to deliver pressurized drugs, i.e., medications, to a thrombus site prior to, during, or after a thrombectomy procedure.

Still another significant aspect and feature of the present invention is the provision and use of an isolation catheter and a jet assembly to deliver pressurized drugs, i.e., medications, to a thrombus site prior to, during, or after a thrombectomy procedure.

Having thus briefly described embodiments of the present invention and having mentioned some significant aspects and features of the present invention, it is the principal object of the present invention to provide an isolation thrombectomy catheter system.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is a view of an isolation thrombectomy catheter system, the present invention;

FIG. 2 is a semi-exploded view of the isolation thrombectomy catheter system of FIG. 1;

FIG. 3 is a cross section view of a manifold and several components connected thereto;

FIG. 4 shows a self-sealing hemostatic seal;

FIG. 5 is a cross section view along line 5-5 of FIG. 3;

FIG. 6 illustrates the alignment of FIGS. 7a and 7b;

FIGS. 7a and 7b together illustrate a cross section view along line 7a,7b-7a,7b of FIG. 1;

FIG. 8 is a cross section view along line 8-8 of FIG. 7b;

FIG. 9 is a cross section view of the distal end of a catheter tube showing a distal occlusive balloon being inflated;

FIG. 10 is an isometric view of a fluid jet emanator;

FIG. 11 is a cross section view of the greater portion of a drug delivery device;

FIG. 12 illustrates the distal end of the catheter tube of the isolation catheter positioned for use in a blood vessel;

FIG. 13 is a cross section view in partial cutaway showing the mode of operation of the isolation thrombectomy catheter system in the performance of the method of the present invention;

FIG. 14 illustrates the jet assembly repositioned proximally within the isolation catheter to interact in the removal of thrombus through one or more of the successive window orifices;

FIG. 15 shows another use of the isolation thrombectomy catheter system where the jet assembly is advanced distally to position the partial length jet catheter partially through the distal end of the isolation catheter to be utilized as a thrombectomy catheter while being afforded a proximally located double seal; and,

FIG. 16 illustrates a drug delivery device in use with isolation catheter to deliver drugs, i.e., medications, to a site in a blood vessel having thrombus.

### DETAILED DESCRIPTION OF THE PREFERRED EMBODIMENTS

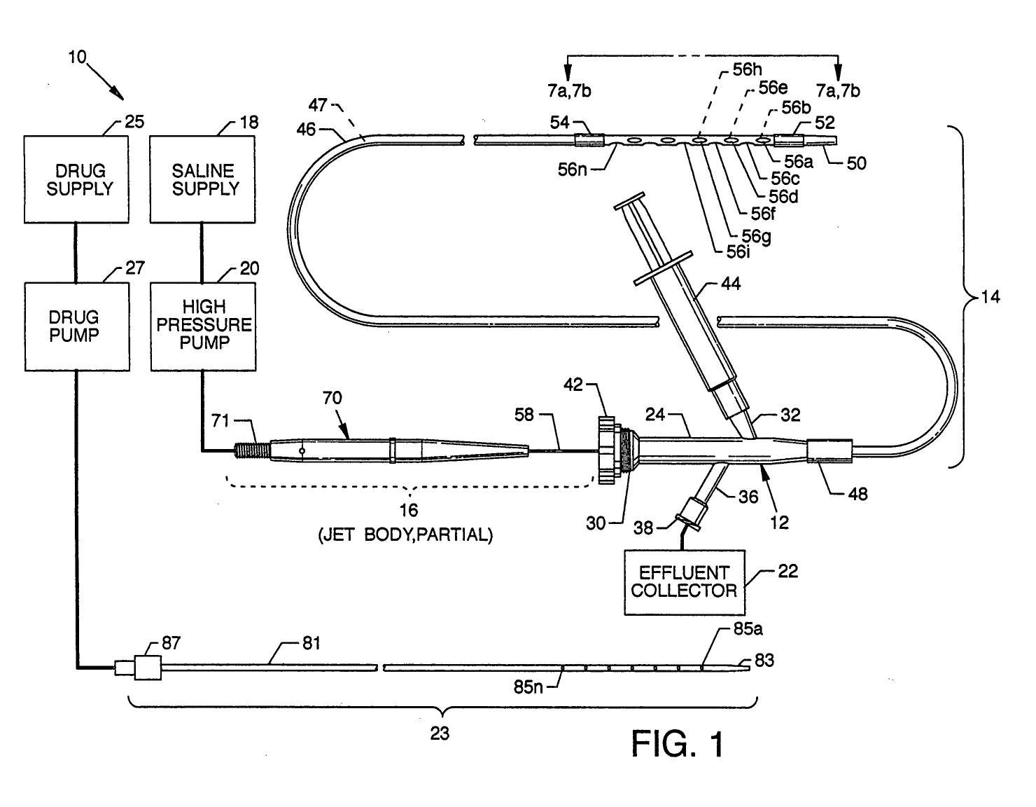

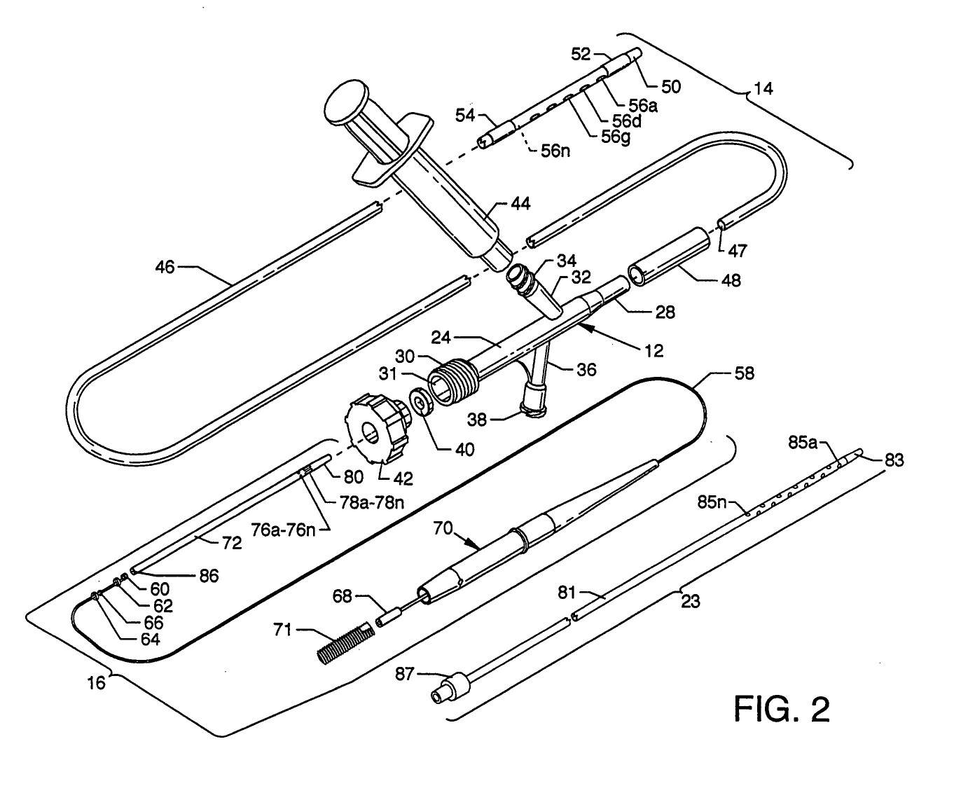

FIG. 1 is a view of the isolation thrombectomy catheter system 10, the present invention, and FIG. 2 is a semi-exploded view of the isolation thrombectomy catheter system 10 of FIG. 1. An initial understanding of the construction and features of the present invention is best made by referring to both FIGS. 1 and 2. Readily observed major components or assemblies of the present invention include a centrally located manifold 12, an isolation catheter 14 which includes a catheter tube 46 and distal and proximal occlusive balloons 52 and 54, a jet assembly 16 (see most completely in FIG. 2), a saline supply 18 and a high pressure pump 20 connected to one end of the jet assembly 16, an effluent collector 22, a drug delivery device 23, and a drug supply 25 and a drug pump 27 connected to one end of the drug delivery device 23. The drug pump 27 is incorporated to infuse drugs or medications at low pressures at a thrombus site prior to or after a thrombectomy procedure while incorporating the isolation catheter 14 and the drug delivery body 23. The high pressure pump 20 can operate over pressure ranges from 200 to 20,000 psi to supply high pressure medium such as saline for ablation purposes at a thrombus site and, alternatively, can be incorporated to inject drugs or medications at high pressure to a thrombus site prior to, during, or after a thrombectomy procedure while incorporating the jet assembly 16.

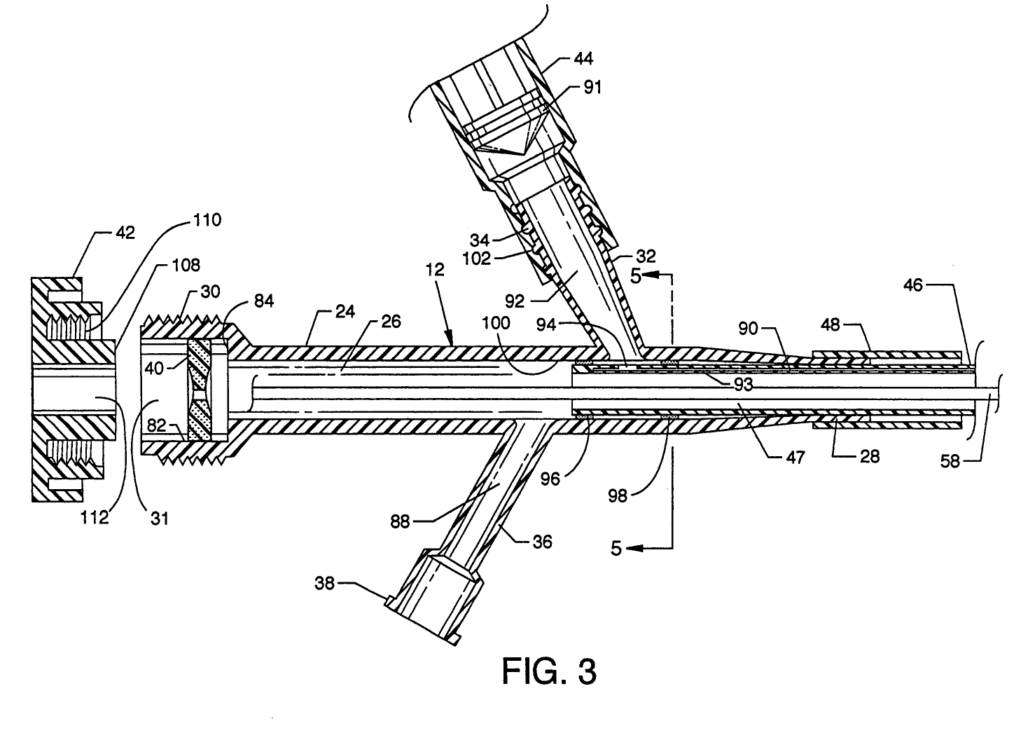

The manifold 12 includes a main body 24, being tubular, a central lumen 26 (FIG. 3) extending a greater portion of the length of the main body 24, a distally located reduced radius end 28, a proximally located threaded end 30, a circular cavity 31 located interiorly of the threaded end 30 in communication with the central lumen 26, an inflation port 32 connected to and extending preferably at an angle from the main body 24, threads 34 at one end of the inflation port 32, an effluent port 36 connected to and extending preferably at an angle from the main body 24, and a threaded flange 38 for accommodation of a Luer connector at one end of the effluent port 36. A pliable and flexible self-sealing hemostatic seal 40, previously disclosed in patent application Ser. No. 10/455,096 aligns in the circular cavity 31. A hemostatic nut 42 aligns to the threaded end 30 of the manifold 12 to compress the hemostatic seal 40 to effect a seal at the proximal end of the manifold 12. Such a seal is effected against a high pressure tube and guidewire, as required. The internal threads of an inflation syringe 44 are accommodated by the threads 34 of the inflation port 32 for connection of the inflation syringe 44 to the manifold.

The isolation catheter 14 includes a flexible catheter tube 46 which can be made of a suitable flexible plastic, such as, but not limited to, PEBAX, polyethylene, polyurethane or braided polyimide; or in the alternative, a flexible mesh can be included in the walls of the flexible catheter tube 46 in combination with the flexible plastic material. The proximal end of the catheter tube 46 is accommodated by and affixed within the central lumen 26 of the manifold 12, as shown in FIG. 3. A strain relief 48 secures over and about the reduced radius end 28 of the manifold 12 and protectingly aligns over and about the catheter tube 46 near the proximal end of the catheter tube 46. The distal end of the catheter tube 46 includes a decreasing radius tapered tip 50 which is flexible. An uninflated low profile distal occlusive balloon 52, which is flexible and has suitable expansion qualities and is made of a suitable material, such as, but not limited to, isoprene, silicone, C-flex, polyurethane, latex, and the like, is located and secured to the catheter tube 46 adjacent to and proximal of the tapered tip 50. An uninflated low profile proximal occlusive balloon 54 having the same qualities as the distal occlusive balloon 52 is located and secured to the catheter tube 46 even more proximal to the tapered tip 50. A plurality of window orifices 56a-56n are located at different locations in the distal region of the catheter tube 46 and dispersed along and about the portion of the catheter tube 46 between the distal occlusive balloon 52 and the proximal occlusive balloon 54. The window orifices 56a-56n extend through the wall of the catheter tube 46 in communication with a centrally located lumen 47 of the catheter tube 46.

The major portion of the jet assembly 16 which is positionable is accommodated in substantially coaxial fashion by the isolation catheter 14, as well as by the manifold 12, as later described in detail. The jet assembly 16 is defined herein as all the components which are moved, aligned, actuated, rotated or otherwise positioned with respect to the isolation catheter 14, the manifold 12, and closely associated components thereof. In particular, the jet assembly 16 includes a high pressure tube 58 having a distally located fluid jet emanator 60, distally located radiopaque marker bands 62 and 64, a distally located support band 66, a ferrule 68 at the proximal end of the high pressure tube 58, a proximally located manual actuator or manual actuator means 70, a proximally located threaded high pressure connector 71, and a partial length jet catheter 72 having a lumen 86, a plurality of outflow orifices 76a-76n, a plurality of inflow orifices 78a-78n, and a flexible tip 80. The high pressure tube 58 and the partial length jet catheter 72 together constitute elongated tubular means.

The drug delivery device 23 includes a drug delivery catheter 81 having at the distal end a tapered tip 83, a plurality of drug dispensing ports 85a-85n located around and about the distal portion, but not including, tapered tip 83, of the drug delivery catheter 81, and a connector 87 at the proximal end. Alternatively, drugs or medications can be delivered to a thrombus site incorporating the jet assembly 16, as previously described.

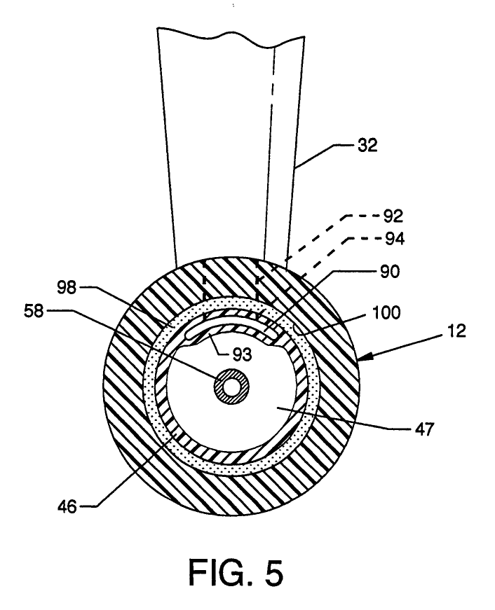

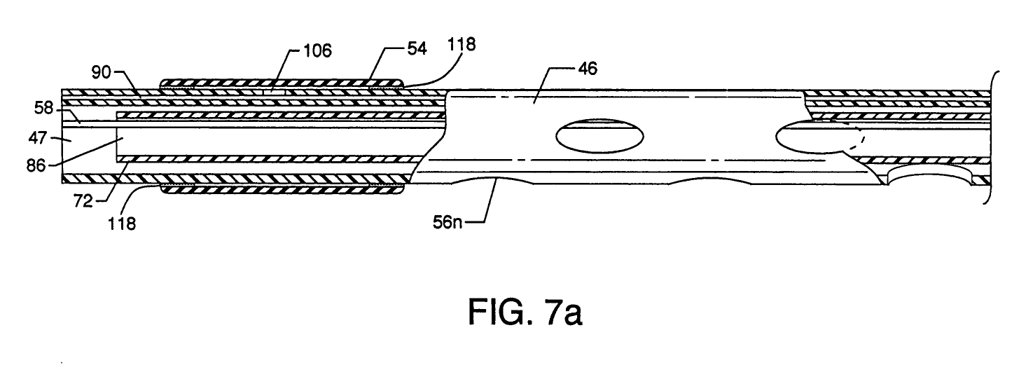

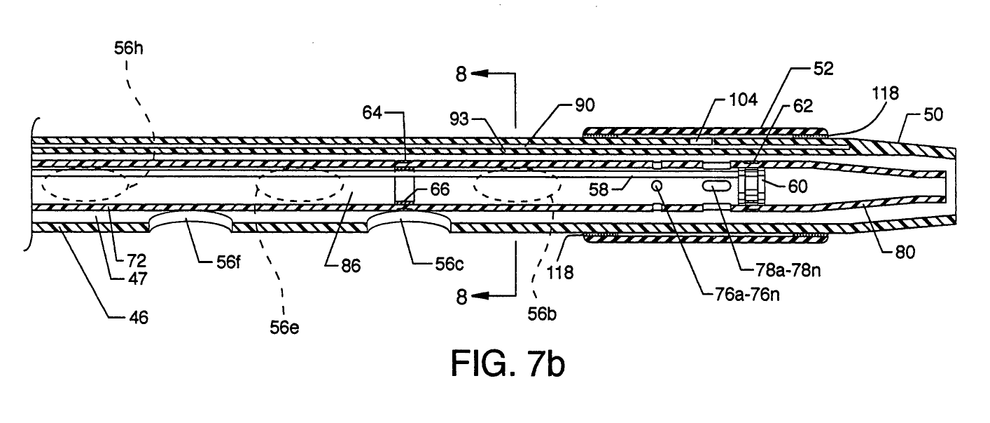

FIG. 3 is a cross section view of the manifold 12 and several components connected thereto. Central to the manifold 12 is the central lumen 26 extending along a greater portion of the length of the manifold 12 to communicate with the proximally located circular cavity 31. The circular cavity 31 is tubular in shape, including a tubular cavity wall 82 and a planar surface 84 which is circular and which intersects the tubular cavity wall 82. The proximal end of the catheter tube 46 aligns and suitably secures within and along the reduced radius end 28 located at the distal portion of the manifold 12 and extends proximally along the central lumen 26 of slightly larger radius (with respect to the reduced radius end 28) slightly beyond the inflation port 32. The catheter tube 46 includes multiple lumens extending in parallel fashion along the length thereof. Each of the separate lumens connects to different and separate portions of the manifold 12, as described herein, to perform different and separate functions. The centrally located lumen 47 provides a passageway for the jet assembly 16 excluding, of course, the manual actuator 70 and other components of the jet assembly 16 proximal to the manual actuator 70, as well as provides a passageway for accommodation of a guidewire. The centrally located lumen 47 connects to and communicates with the central lumen 26 of the manifold 12. The centrally located lumen 47 also communicates with a lumen 88 of the effluent port 36 for exhausting of effluent passing proximally along the catheter tube 46. An inflation lumen 90, as also shown in FIG. 5, and having a low profile, is located within and extends along a thick wall region 93 of the catheter tube 46. Saline, saline with a contrast fluid, or a gas such as carbon dioxide, or other suitable gas can be appropriately introduced into the inflation port 32 to pass through the inflation lumen 90 to inflate the distal and proximal occlusive balloons 52 and 54 shown in FIGS. 7a and 7b. The distal portion of the inflation lumen 90 connects and communicates with the distal and proximal occlusive balloons 52 and 54, as shown in FIGS. 7a and 7b, and the proximal portion of the inflation lumen 90 communicates and connects with a lumen 92 of the inflation port 32 through a proximally located inflation orifice 94 extending through the thick wall region 93 of the catheter tube 46 from the inflation lumen 90. The inflation orifice 94 preferably is located in close proximity to the lumen 92 of the inflation port 32. Such communication requires that the inflation orifice 94 be sealed to the lumen 92 for dedicated communication therebetween and, as such, this is accomplished by the use of annular opposed seals 96 and 98 extending in annular fashion between the catheter tube 46 and the circular interior wall 100 of central lumen 26 and flanking the inflation orifice 94. The seals 96 and 98 can be of suitable composition, whereby a suitable seal can be achieved. For example, the seals 96 and 98 can be adhesive or could be of other material, such as plastic or metal, incorporated in frictional engagement to provide a suitable seal. Sealing accomplished in this or any other suitable fashion ensures dedicated communication of the inflation orifice 94 with the lumen 92 of the inflation port 32, as well as providing for separation of the catheter tube lumen 47 from the inflation port lumen 92. Also shown are threads 102 located internally at the lower region of the inflation syringe 44 in engagement with the external threads 34 of the inflation port 32, thereby providing for connection and ensuring communication between the inflation syringe 44, including the plunger 91, and the lumen 92 of the inflation port 32. Such communication also extends along and between the lumen 92, the inflation orifice 94, the inflation lumen 90, a distal occlusive balloon inflation orifice 104 (FIG. 7b) and distal occlusive balloon 52, and a proximal occlusive balloon inflation orifice 106 (FIG. 7a) and proximal occlusive balloon 54. Hemostatic nut 42 is influential in sealing the proximal end of the manifold 12 and includes a centrally located cylindrical boss 108 and interior threads 110, the latter being provided for engagement of the threaded end 30 of the manifold 12. Tightening of the hemostatic nut 42 forces the cylindrical boss 108 to compress the hemostatic seal 40 against the planar surface 84 of the circular cavity 31, to expandingly and sealingly force the hemostatic seal 40 into intimate and forced contact with the cavity wall 82 and to compressingly and sealingly force the hemostatic seal 40 inwardly against the high pressure tube 58 and/or against a guidewire (not shown) if present. A centrally located passage 112 is located along the centerline of the hemostatic nut 42 for passage of the high pressure tube 58 and a guidewire.

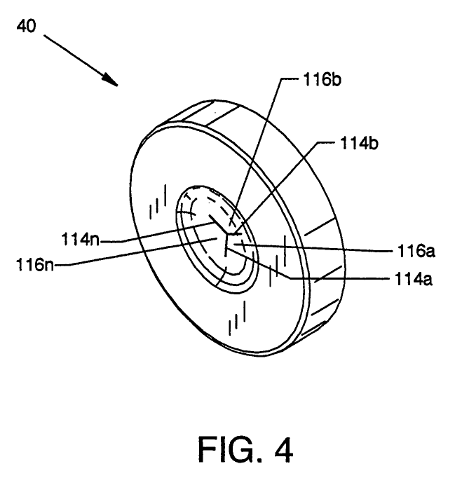

FIG. 4 shows the self-sealing hemostatic seal 40 of medical grade silicone material, described in detail in patent application Ser. No. 10/455,096. The medical grade silicone material is parted or otherwise separated to form a plurality of slits 114a-114n, each slit extending outwardly in radial fashion from the center of the self-sealing hemostatic seal 40 creating boundaries beneficial in defining lobes 116a-116n. That is to say that lobe 116a is located between slits 114a and 114b, lobe 116b is located between slits 114b and 114n, and lobe 116n is located between slits 114n and 114a. Adjacent lobes 116a-116n are in mutual contact along the slits 114a-114n to effect a seal from the proximal side to the distal side of the self-sealing hemostatic seal 40. The diameter of the self-sealing hemostatic seal 40 is slightly larger than that of the circular cavity 31 of the manifold 12 to provide for flexible but snug frictional engagement of the self-sealing hemostatic seal 44 within the circular cavity 31, as well as to provide for circumferential sealing of the self-sealing hemostatic seal 40 to the circular cavity 31.

FIG. 5 is a cross section view along line 5-5 of FIG. 3 showing the relationship of the inflation lumen 90 of the catheter tube 46 to the centrally located lumen 47 of the catheter tube 46, as well as showing the relationship of the catheter tube 46 to the manifold 12 and seal 98. The cross section of the inflation lumen 90 is arcuate in shape and is extendingly projected along the length of and within a thick wall region 93 of the catheter tube 46. The structure of the arcuate and low profile inflation lumen 90 provides for minimal protrusion, intrusion and interference with the generally round cross section of the lumen 47 to allow the size of the partial length jet catheter 72 to be maximized to be able to occupy the greatest amount of space within the lumen 47 of the catheter tube 46. Maximizing the size of the partial length jet catheter 72 is beneficial to increased operating efficiency of the invention during thrombectomy procedure, wherein the partial length jet catheter 72, including the fluid jet emanator 60, is located in close and suitable proximity to the window orifices 56a-56n of the catheter tube 46.

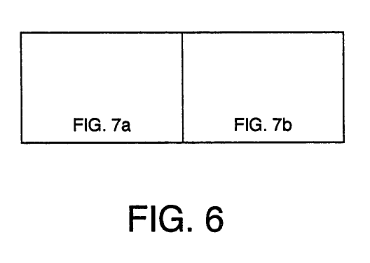

FIG. 6 illustrates the alignment of FIGS. 7a and 7b.

FIGS. 7a and 7b together illustrate a cross section view along line 7a,7b-7a,7b of FIG. 1 showing the relationship of the partial length jet catheter 72 subsequent to advancement along the lumen 47 of the catheter tube 46 to the distal location in the catheter tube 46. Shown in particular is the distal occlusive balloon 52 being located and secured around, over and about the distal end of the catheter tube 46 and the proximal occlusive balloon 54 located and secured around, over and about a more proximal location of the catheter tube 46 such as by adhesive 118 located between the inner surfaces of the balloon ends and the catheter tube 46. The distal occlusive balloon 52 is flexible, has suitable expansion qualities, and is made of a suitable material, such as, but not limited to, polyurethane, isoprene, silicone, C-flex, latex, and the like. The distal occlusive balloon 52 is also aligned over the distal occlusive balloon inflation orifice 104 which extends through the outer portion of the thick wall region 93 of the catheter tube 46, thereby providing connection and communication between the sealed-off interior portion of the distal occlusive balloon 52 and the inflation lumen 90. In a like manner, the similarly constructed proximal occlusive balloon 54 aligns and secures over the proximal occlusive balloon inflation orifice 106 which extends through the outer portion of the thick wall region 93 of the catheter tube 46, thereby providing connection and communication between the sealed-off interior portion of the proximal occlusive balloon 54 and the inflation lumen 90. The distal and proximal occlusive balloons 52 and 54 are in common communication through the portion of the inflation lumen 90 extending therebetween and beyond and, as such, pressurized inflation medium in the inflation lumen 90 applies a common pressure to the distal and proximal occlusive balloons 52 and 54 to inflate them simultaneously. In the alternative, more than one inflation lumen could be provided to exercise separate inflation and control of the occlusive balloons.

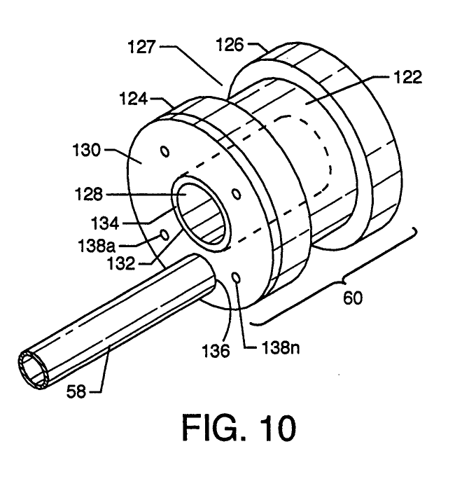

The partial length jet catheter 72 secures to the high pressure tube 58 by any of several methods. One such method involves welding or otherwise suitably attaching part of the high pressure tube 58 to the inner surface of the support band 66 and then engaging the radiopaque marker 64 in close intimate frictional engagement over the partial length jet catheter 72 to forcibly compress the partial length jet catheter 72 against the support band 66. Another fastening method involves another radiopaque marker 62 engaged in close intimate frictional engagement over the partial length jet catheter 72 to force the partial length jet catheter 72 against the fluid jet emanator 60. The structure of the fluid jet emanator 60, as described in detail with reference to FIG. 10, accommodates the overlying depressed partial length jet catheter 72.

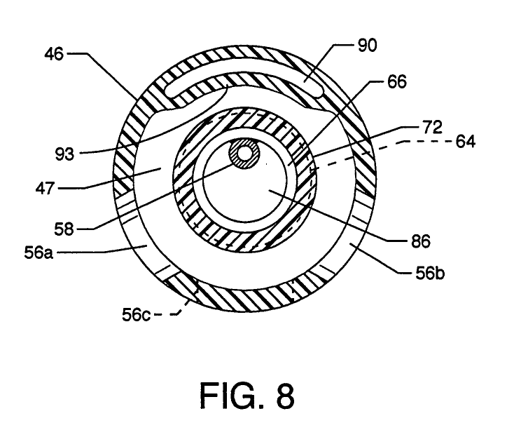

FIG. 8 is a cross section view along line 8-8 of FIG. 7b. Shown in particular are the window orifices 56a-56n distributed about the longitudinal centerline of the catheter tube 46 and extending through the wall of the catheter tube 46 where the window orifices 56a-56n are shown at locations which do not conflict with the thick wall region 93 of the catheter tube 46 or the inflation lumen 90. In the view, a series of window orifices 56b, 56e, 56h . . . are located at the four o'clock position, a series of window orifices 56c, 56f, 56i . . . are located at the six o'clock position (dashed lines), and a series of window orifices 56a, 56d, 56g . . . are located at the eight o'clock position to form three rows of successively staggered and alternatingly located window orifices 56a-56n, as partially seen in FIGS. 7a-7b. Although three rows of successively alternatingly located window orifices 56a-56n are described, other numbers of rows may be incorporated or other arrangements, positions, relationships and distributions of the window orifices 56a-56n may be utilized according to the teachings of the invention.

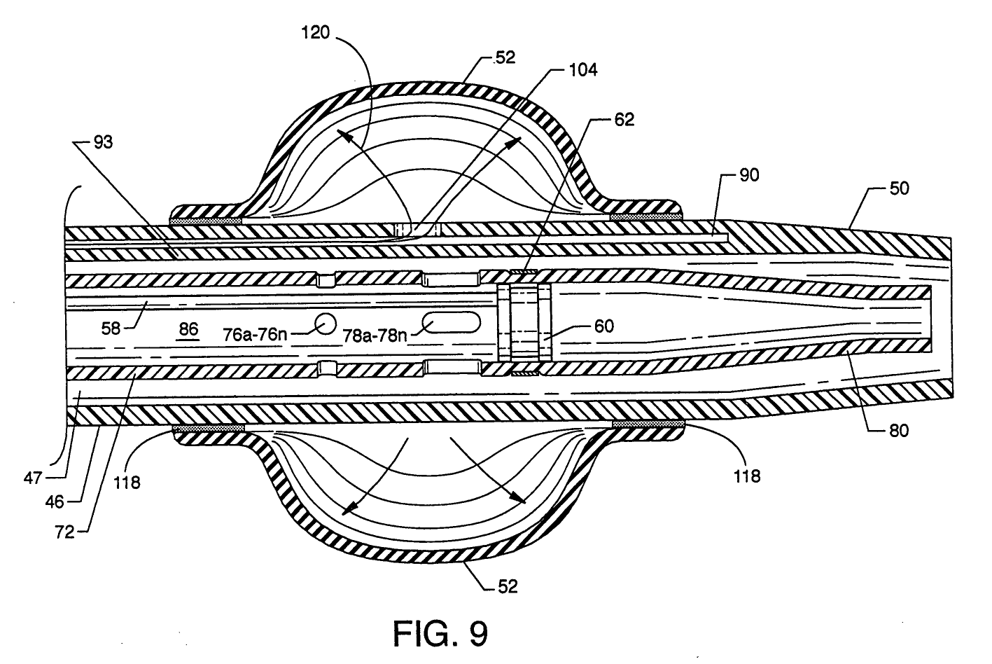

FIG. 9 is a cross section view of the distal end of the catheter tube 46 showing the distal occlusive balloon 52 being inflated. Inflation medium 120, such as saline, contrast fluid, a combination of saline and contrast fluid, gas, carbon dioxide, or other suitable medium, is forced along the inflation lumen 90 by action of the inflation syringe 44 and through the distal occlusive balloon inflation orifice 104 to the interior of the distal occlusive balloon 52 to outwardly expand the distal occlusive balloon 52. While both liquid and gaseous inflation media can be used, the use of a gaseous inflation medium allows the use of a smaller size inflation lumen. The distal occlusive balloon 52 can be compliant, semi-compliant, or non-compliant.

FIG. 10 is an isolation view of the fluid jet emanator 60, previously described in detail as a jet cap and fully disclosed in patent application Ser. No. 09/888,455, which is utilized at the distal end of the high pressure tube 58 of the jet assembly 16. The readily visible components and main features of the fluid jet emanator 60 include a cylindrical-like main body 122 having opposing annular rings 124 and 126 extending at the ends thereof, a guidewire lumen 128 extending through the main body 122, a round plate 130 including a central hole 132, an annular extension 134, a receptor hole 136 in the round plate 130 for accommodation of the distal end of the high pressure tube 58 and a plurality of rearwardly directed jet orifices 138a-138n aligned concentrically to the central hole 132. The fluid jet emanator 60 provides structure in the form of an annular groove 127 between the annular rings 124 and 126 suitably adapted for secure engagement of the partial length jet catheter 72 to the fluid jet emanator 60. The annular groove 127 accommodates the surface of the partial length jet catheter 72 as depressed by the radiopaque marker band 62. Alternatively, in emanator devices, as described in patent applications by the assignee, the distal end of the high pressure tube 58 can be formed in many geometric configurations and can include a plurality of jet orifices to form emanator structure suitable for use as a fluid jet emanator having rearwardly directed fluid jets which can be incorporated into use with the present invention. The use of the fluid jet emanator illustrated in FIG. 10 shall not be deemed to be limiting to the scope of the invention; other fluid jet emanators can be used.

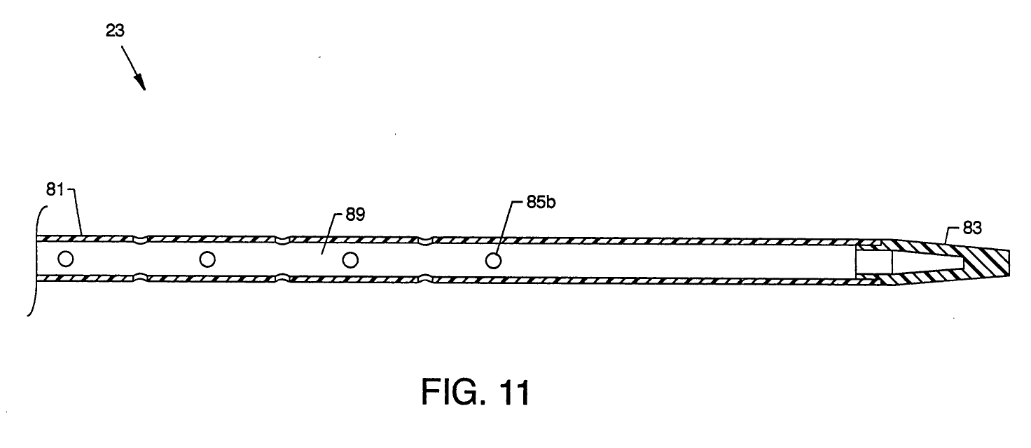

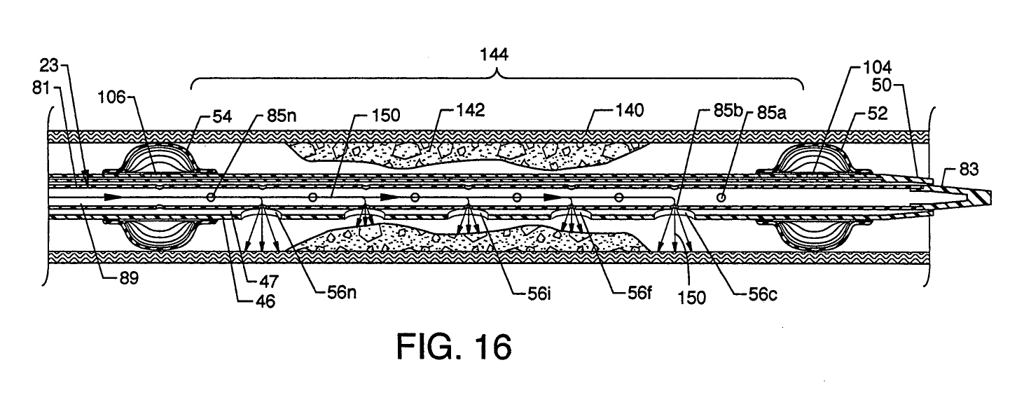

FIG. 11 is a cross section view of the greater portion of the drug delivery device 23. The drug delivery device 23 is fashioned of a suitable flexible plastic material and is incorporated for dispensing drugs at lower pressures of a 200 to 20,000 psi range or may be fashioned of a metal hypo-tube for dispensing drugs at high pressures of a 200 to 20,000 psi range. The drug delivery device 23 includes a lumen 89 communicating with the plurality of drug dispensing ports 85a-85n for the introduction of drugs or medications and the like for lysing or other treatment of thrombus, as described in detail with reference to FIG. 16. Also shown is the tapered tip 83 fixedly engaging the distal end of the drug delivery catheter 81. Provision of the tapered tip 83 enables easy navigation through the isolation catheter 14.

### MODE OF OPERATION

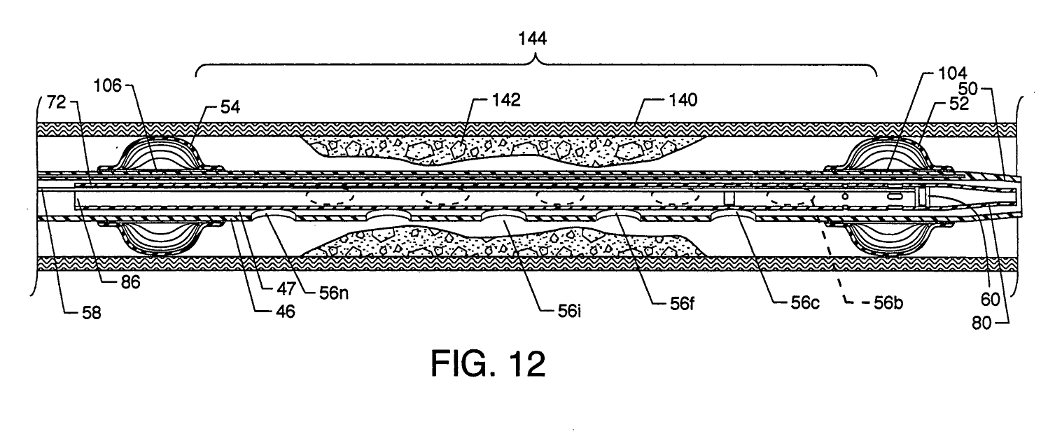

FIG. 12 illustrates the distal end of the catheter tube 46 of the isolation catheter 14 positioned for use in a blood vessel 140. Fluoroscopy or other such suitable methods are incorporated to view the distal end of the catheter tube 46 and the distal end of the jet assembly 16 during placement. A guidewire (not shown) is utilized to guide the distal end of the isolation catheter 14 and the distal end of the jet assembly 16 to the site of the thrombus 142. In general, and for the purpose of discussion, thrombotic material, clots, lesions, and the like are referred to as thrombus 142, unless otherwise noted. Preferably, the distal end of the isolation catheter 14 is advanced to position the uninflated distal occlusive balloon 52 just distal of the thrombus 142 and to place the uninflated proximal occlusive balloon 54 just proximal of the thrombus 142. The distal occlusive balloon 52 and the proximal occlusive balloon 54 are then inflated by using inflation syringe 44 to deliver inflation medium through lumen 92 of inflation port 32 to inflation lumen 90 by way of inflation orifice 94 and thence through orifices 104 and 106 at the distal end of the catheter tube 46 so that the distal occlusive balloon 52 and the proximal occlusive balloon 54 are placed into contact with and seal against the blood vessel 140. Such inflation establishes a sealed region 144 extending along the interior of the blood vessel 140 from the annular contact of the inflated distal occlusive balloon 52 with the blood vessel 140 to the annular contact of the inflated proximal occlusive balloon 54 with the blood vessel 140, and also along and between the distal exterior region of the catheter tube 46 extending between the inflated distal occlusive balloon 52 and the inflated proximal occlusive balloon 54 and the blood vessel 140. Such a sealed region 144 of occlusion contains and seals the deposits of thrombus 142 about the exterior of the included sealed distal end of the catheter tube 46 and the interior of the blood vessel 140 extending between the distal occlusive balloon 52 and the proximal occlusive balloon 54 and ensures removal of broken-up and macerated thrombotic deposits or lesions through the window orifices 56a-56n, as well as prevents migration of broken-up and macerated thrombotic deposits or lesions along the vasculature during thrombectomy procedures. Such an arrangement is helpful in preventing proximal and distal embolizations. Inflation of the inflatable distal and proximal occlusive balloons 52 and 54 provides for centering of the catheter tube 46 within the blood vessel 140 to provide for centrally located and evenly applied saline emanation which can also preclude having the jetted saline emitted dangerously close to the wall of the blood vessel 140. Such centering allows for more powerful suction within the lumen 47 of the catheter tube 46 without damage to the wall of the blood vessel 140.

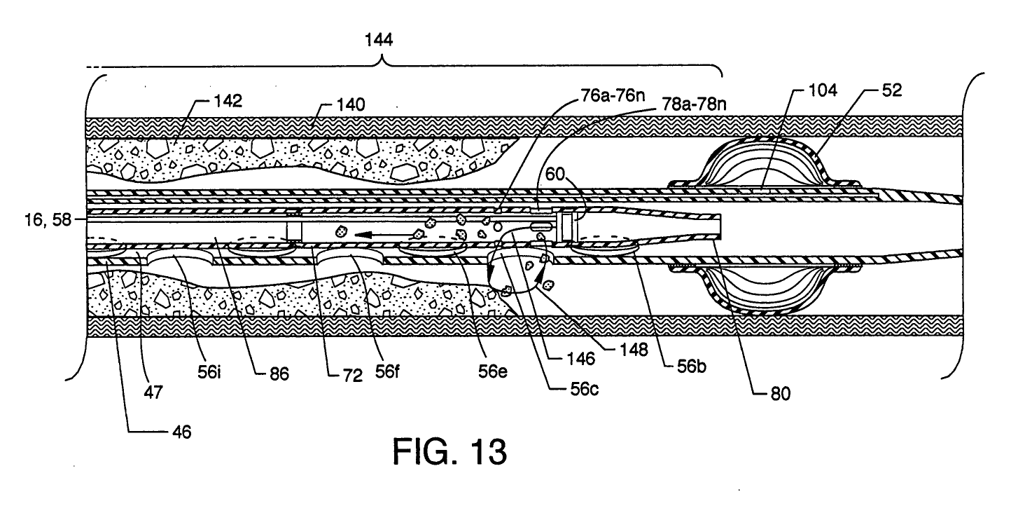

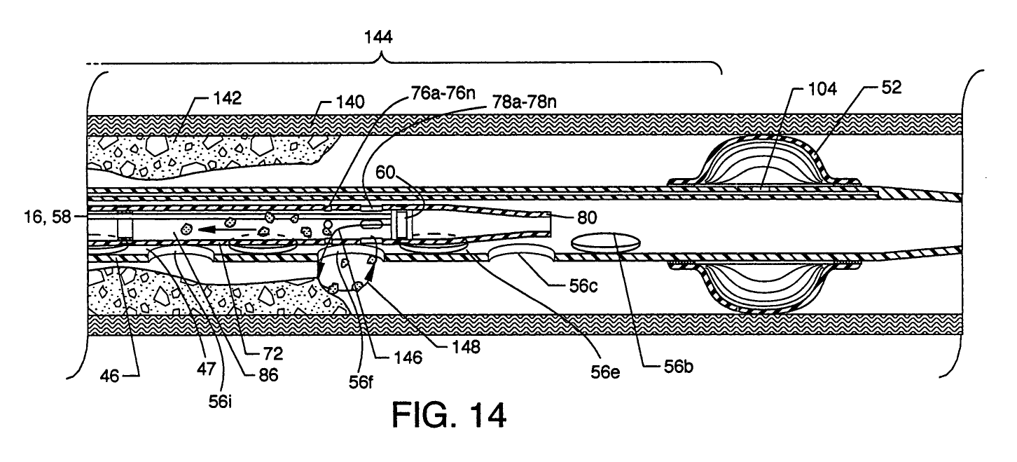

FIG. 13 is a cross section view in partial cutaway showing the mode of operation of the isolation thrombectomy catheter system 10 in the performance of the method of the present invention; and FIG. 14 illustrates the jet assembly 16 repositioned proximally within the isolation catheter 14 to interact in the removal of thrombus through one or more of the successive window orifices 56a-56n. Illustrated in FIG. 13 is the breakup, dislodging, maceration and exhausting of thrombus 142 by action of one or more fluid jets 146 to cause cross stream action directed through the window orifice 56c with attention to the distal end of the jet assembly 16, particularly the fluid jet emanator 60, where the fluid jet emanator 60 is positioned to achieve cross stream action directed through the window orifice 56c. One or more high velocity fluid jets 146 of saline (or other suitable fluid) is shown being emitted in a proximal direction from the fluid jet emanator 60 to provide for cross stream action to break-up, dislodge, and macerate thrombus 142 and to impinge upon and carry away thrombus 142. Other fluid jet emanators can be incorporated at the distal end of the jet assembly 16 as an alternative to the fluid jet emanator 60 to emanate or emit one or more high velocity fluid jets 146 proximally along or near the longitudinal axis of the high pressure tube 58 and subsequently the catheter tube 46 to accomplish the same purpose as that described for the fluid jet emanator 60. The high velocity fluid jet(s) 146 of saline pass outwardly through the outflow orifice(s) 76a-76n in a radial direction creating cross stream jet(s) 148 (lower velocity jet(s)) directed outwardly toward the wall of the blood vessel 140 and are influenced by the low pressure at the inflow orifice(s) 78a-78n to cause the cross stream jet(s) 148 to flow circumferentially and distally to impinge on, provide drag forces on, and break up deposits of thrombus 142 and to, by entrainment, urge and carry along the particles of thrombotic deposits or lesions 142 through the inflow orifice(s) 78a-78n, a relatively low pressure region, into the high velocity fluid jets 146 where the thrombus 142 is further macerated into microscopic particles, into the lumen 86 of the partial length jet catheter 72, and thence into the lumen 47 of the catheter tube 46 for exhausting therethrough. The entrainment through the inflow orifice(s) 78a-78n is based on entrainment by the high velocity fluid jets 146. The outflow is driven by internal pressure which is created by the high velocity fluid jets 146 and the fluid entrained through the inflow orifice(s) 78a-78n. Enhanced clot removal is attainable because of the recirculation pattern established between inflow and outflow orifices 78a-78n and 76a-76n, which creates a flow field that maximizes drag force on wall-adhered thrombus 142. Since the entrained thrombus is macerated into microscopic particles, those particles that exit the outflow orifices 76a-76n are not of sufficient size to significantly block the distal circulation, and will be re-entrained into the inflow orifices 78a-78n at a high rate. During the thrombectomy procedure, the jet assembly 16 can be rotated about its longitudinal axis by means of the manual actuator 70, thereby offering 3600 capability to completely remove thrombus from the wall of the blood vessel 140 in radial fashion.

Subsequent to successful ablation and carrying away of thrombus 142, as shown and described with reference to FIG. 13, the jet assembly 16, is repositioned proximally with the manual actuator 70 to interact in the removal of thrombus 142 through one or more of the successive window orifices 56a-56n, such as window orifice 56e, and repeatedly and subsequently repositioned proximally with the manual actuator 70 to interact in the removal of thrombus 142 through the next window orifice 56f, as shown in FIG. 14, and then repositioned proximally in repetition to the remaining window orifices 56a-56n and activated until all the thrombus 142 within the sealed region 144 has been successfully ablated and removed. Such repositioning of the jet assembly 16 by the manual actuator 70 can occur either proximally or distally in back and forth motion, if desired, along with simultaneous rotation of the jet assembly 16 to effect full coverage ablation. Effluent in the form of broken-up and macerated thrombus 142 is delivered through the lumen 47 of the catheter tube 46 to the effluent port 36 and delivered to the effluent collector 22. Optionally, the output of the effluent collector 22 may be regulated to control the effluent discharge rate and for the purpose of other system functions. Subsequent to a successful thrombectomy procedure, operation of the high pressure pump 20 supplying high pressure saline is terminated, the distal occlusive balloon 52 and the proximal occlusive balloon 54 are deflated, and then removal of the jet assembly 16 and the isolation catheter 14 can be effected.

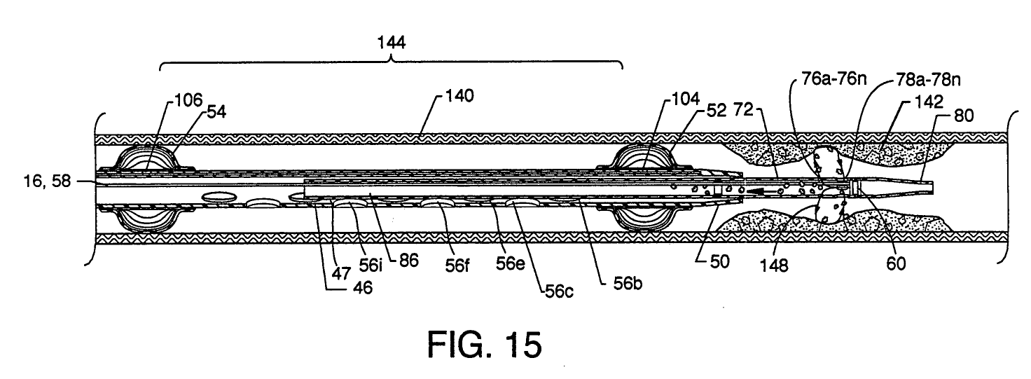

FIG. 15 shows another use of the isolation thrombectomy catheter system 10 where the jet assembly 16 is advanced distally to position the partial length jet catheter 72 partially through the distal end of the isolation catheter 14 to be utilized as a thrombectomy catheter while being afforded a proximally located double seal as provided by the distal occlusive balloon 52 and the proximal occlusive balloon 54. The catheter tube 46 of the isolation catheter 14, and thus the partial length jet catheter 72, is centralized in the blood vessel 140 by the inflation of the distal occlusive balloon 52 and the proximal occlusive balloon 54, thereby substantially maintaining an equal distance between the outflow orifices 76a-76n and inflow orifices 78a-78n and the wall of the blood vessel 140. Such centralized location of the partial length jet catheter 72 keeps the impingement of the cross stream jets 148 from undesirable closer and more intimate and potential wall damaging contact with the walls of the blood vessel 140, as well as provides for equal distance radial impingement of the thrombus 142 by the cross stream jets 148. As previously described, the jet assembly 16 can be positioned with the manual actuator 70 longitudinally either proximally or distally with respect to the isolation catheter 14, as well as rotated about its longitudinal axis for complete ablation of the thrombus 142 shown distal to the tapered tip 50 of the catheter tube 46. The cross stream jets 148 impinge, break-up, and macerate the thrombus 142 which is then exhausted through the lumen 86 of the partial length jet catheter 72 and thence through the lumen 47 of the catheter tube 46.

FIG. 16 illustrates drug delivery device 23 in use with isolation catheter 14 to deliver drugs, i.e., medications, to a site in a blood vessel 140 having thrombus 142. Preferably, the drug delivery device 23, including the tapered tip 83, has a diameter just slightly larger than the inner and smallest diameter of the tapered tip 50 of the catheter tube 46, but still adequately sized to navigate through the catheter tube 46. The drug delivery device 23 is introduced into the manifold 12 and into the catheter tube 46 and advanced distally to position the tapered tip 83 into intimate contact with the inner and narrowest portion of the tapered tip 50 of the catheter tube 46, the mating of the tapered tip 83 with the tapered tip 50 effecting sealing means which seals the distal end of the catheter tube 46 against leakage or migration of drugs (medications) distally. Such positioning places the drug dispensing ports 85a-85n in near proximity to the window orifices 56a-56n of the catheter tube 46. The drug pump 27 urges drugs or medications 150 through the lumen 89 of the drug delivery catheter 81 to be dispensed outwardly from the plurality of drug dispensing ports 85a-85n, whereupon the drugs or medications 150 enter the lumen 47 of the catheter tube 46 to be further dispensed through the window orifices 56a-56n of the catheter tube 46 for intimate contact with the thrombus 142 for lysing. The drugs or medications 150 are contained in the sealed region 144 between the distal occlusive balloon 52 and the proximal occlusive balloon 54. Thrombectomy procedures can be accomplished solely using the jet assembly 16 with the isolation catheter 14 in combination or thrombectomy procedures can be accomplished by alternatingly using the jet assembly 16 and the drug delivery device 23 in combination in a united thrombectomy effort. The isolation catheter 14 and the drug delivery device 23 in combination can be used to deliver drugs or medications 150 to a thrombus site, or the isolation catheter 14 and the jet assembly 16 in combination may be used to deliver drugs or medications 150 to a thrombus site.

Various modifications can be made to the present invention without departing from the apparent scope thereof.

It is claimed:

1. A catheter system comprising:
a. an isolation catheter having a proximal end, a distal end, a central lumen with a wall extending from said proximal end to said distal end, an inflation lumen also extending from said proximal end to said distal end, a proximal balloon, and a distal balloon, said proximal balloon and said distal balloon together defining an isolation portion near the distal end of the isolation catheter, said inflation lumen being in fluid communication with said proximal balloon and said distal balloon to provide for inflation of said proximal balloon and said distal balloon in a body vessel or cavity, thereby to isolate a portion of the body vessel or cavity, said isolation portion having at least one opening through said wall of said central lumen;
b. treatment apparatus comprising elongated tubular means having a proximal end and a distal end and manual actuator means coupled to said elongated tubular means proximate said proximal end, said elongated tubular means having a wall and at least one lumen for passage of fluid between said proximal and distal ends, said wall having at least one opening therethrough proximate said distal end; and,
c. said elongated tubular means being configured so that said manual actuator means can be used to position a portion of said elongated tubular means within said isolation catheter to locate said at least one opening through said wall of said elongated tubular means along said isolation portion so that fluid passing through said at least one opening through said wall of said elongated tubular means interacts with the isolated portion of the body vessel or cavity through said at least one opening through said wall of said central lumen.

2. The catheter system of claim 1, wherein said at least one opening through said wall of said central lumen comprises a plurality of windows.

3. The catheter system of claim 1, further comprising an effluent collector in fluid communication with said central lumen and adapted to receive fluid from said central lumen.

4. An isolation thrombectomy catheter system comprising:
a. an isolation catheter having a proximal end, a distal end, a central lumen with a wall extending from said proximal end to said distal end, and an inflation lumen also extending from said proximal end to said distal end, a proximal balloon, and a distal balloon, said proximal balloon and said distal balloon together defining an isolation portion near the distal end of the isolation catheter, said inflation lumen being in fluid communication with said proximal balloon and said distal balloon to provide for inflation of said proximal balloon and said distal balloon in a body vessel or cavity, thereby to isolate a portion of the body vessel or cavity, said isolation portion having at least one opening through said wall of said central lumen;
b. treatment apparatus comprising elongated tubular means having a proximal end and a distal end and manual actuator means coupled to said elongated tubular means proximate said proximal end, said elongated tubular means having a wall and at least one lumen for passage of fluid between said proximal and distal ends, said wall having at least one opening therethrough proximate said distal end;
c. said elongated tubular means being configured so that said manual actuator means can be used to position a portion of said elongated tubular means within said isolation catheter to locate said at least one opening through said wall of said elongated tubular means along said isolation portion;
d. a fluid source forming part of said treatment apparatus;
e. a fluid pump also forming part of said treatment apparatus; and,
f. said fluid pump being connectible to said fluid source and to said proximal end of said elongated tubular means and being adapted to pump fluid from said fluid source into said elongated tubular means so that the fluid interacts with the isolated portion of the body vessel or cavity through said at least one opening through said wall of said elongated tubular means and said at least one opening through said wall of said central lumen.

5. The isolation thrombectomy catheter system of claim 4, further comprising an effluent collector in fluid communication with said central lumen and adapted to receive fluid from said central lumen.

6. The isolation thrombectomy catheter system of claim 4, further comprising a hemostatic seal which allows movement of said elongated tubular means in said isolation catheter and which minimizes leakage of blood or fluid between said isolation catheter and said elongated tubular means.

7. The isolation thrombectomy catheter system of claim 6, wherein said hemostatic seal is located proximate the proximal end of said isolation catheter.

8. The isolation thrombectomy catheter system of claim 4, further comprising a tapered tip at the distal end of said isolation catheter.

9. The isolation thrombectomy catheter system of claim 4, wherein said at least one opening through said wall of said central lumen comprises a plurality of windows.

10. An isolation thrombectomy catheter system comprising:
a. an isolation catheter having a proximal end, a distal end, a central lumen with a wall extending from said proximal end to said distal end, an inflation lumen also extending from said proximal end to said distal end, a proximal balloon, and a distal balloon, said proximal balloon and said distal balloon together defining an isolation portion near the distal end of the isolation catheter, said inflation lumen being in fluid communication with said proximal balloon and said distal balloon to provide for inflation of said proximal balloon and said distal balloon in a body vessel or cavity, thereby to isolate a portion of the body vessel or cavity, said isolation portion having at least one opening through said wall of said central lumen;
b. treatment apparatus comprising elongated tubular means including a high pressure tube having proximal and distal ends for passage of high pressure fluid between said proximal and distal ends, a fluid jet emanator in fluid communication with said high pressure tube at said distal end, and manual actuator means proximate said proximal end;
c. said high pressure tube and said fluid jet emanator being configured so that said manual actuator means can be used to position said fluid jet emanator within said isolation catheter along said isolation portion;
d. a fluid source forming part of said treatment apparatus;
e. a high pressure fluid pump also forming part of said treatment apparatus; and,
f. said high pressure fluid pump being connectible to said fluid source and to said proximal end of said high pressure tube and being adapted to pump fluid from said fluid source into said high pressure tube at high pressure so that fluid emanates from said fluid jet emanator to create at least one fluid jet which interacts with the isolated portion of the body vessel or cavity through said at least one opening through said wall of said central lumen.

11. Treatment apparatus comprising:
a. a partial length jet catheter having a proximal end and a distal end;
b. a high pressure tube for passage of high pressure fluid, said high pressure tube having proximal and distal ends;
c. a fluid jet emanator in fluid communication with said high pressure tube at said distal end of said high pressure tube and located within said partial length jet catheter;
d. manual actuator means coupled to said proximal end of said high pressure tube, said partial length jet catheter, said high pressure tube, and said fluid jet emanator being configured so that they can be manipulated by said manual actuator means and therewith be positioned within a lumen of an isolation catheter;
e. high pressure connection means at the proximal end of said high pressure tube connectible to a source of high pressure fluid; and,
f. said fluid jet emanator being configured to create at least one fluid jet when said high pressure connection means is connected to a source of high pressure fluid.

12. The treatment apparatus of claim 11, wherein said partial length jet catheter has a short effluent lumen having a proximal end and a distal end and providing for flow of effluent fluid from said distal end to said proximal end so that effluent fluid and debris passes from said proximal end of said short effluent lumen proximally along the lumen of the isolation catheter.

13. The treatment apparatus of claim 11, wherein said partial length jet catheter has at least one inflow orifice and at least one outflow orifice, said at least one outflow orifice creating cross stream jets when said fluid jet emanator creates said at least one fluid jet, said cross stream jets being locatable to treat an isolated portion of a body vessel through openings in the isolation catheter.

14. An isolation catheter for isolating a portion of a body vessel or cavity comprising:
a. an elongated device having a proximal end and a distal end and having at least one balloon inflation lumen and a central lumen extending along the length thereof;
b. a proximal balloon and a distal balloon mounted on said elongated device near said distal end and spaced apart and defining an isolation portion of said elongated device therebetween;
c. said balloon inflation lumen being in fluid communication with said proximal balloon and said distal balloon to provide for inflation and deflation of said proximal balloon and said distal balloon in a body vessel or cavity, said proximal balloon and said distal balloon being expandable upon inflation to seal between said elongated device and the walls of the body vessel or cavity, thereby to isolate a portion of the body vessel or cavity at said isolation portion;
d. said central lumen having a wall and at least one opening through said wall at said isolation portion, said central lumen being adapted to receive treatment apparatus;
e. said at least one opening providing fluid communication between said central lumen and the body vessel or cavity so that said treatment apparatus when positioned adjacent to said at least one opening, has fluid communication with the body vessel or cavity to effect treatment of the body vessel or cavity while said proximal balloon and said distal balloon are inflated thereby isolating a portion of the body vessel or cavity.

15. The isolation catheter of claim 14, further comprising sealing means at the distal end of said central lumen to reduce leakage of fluid between said distal end of said central lumen and a distal portion of said treatment apparatus when disposed therein.

16. The isolation catheter of claim 15, wherein said sealing means comprises a tapered distal tip, said tapered distal tip having an inner diameter comprising a distal portion of said central lumen which tapers to form a short portion of close or interference fit against an external distal portion of said treatment apparatus when disposed therein.

17. A method of treating a segment of a blood vessel or other body cavity, comprising the steps of:
a. providing an isolation catheter having a central lumen, an inflation lumen, a proximal balloon, a distal balloon, and an isolation portion between the proximal balloon and the distal balloon, the isolation portion having at least one opening through a wall of the central lumen;
b. introducing the isolation catheter into a blood vessel or other body cavity and positioning the isolation catheter at a portion of the blood vessel or other body cavity to be treated;
c. inflating the proximal balloon and the distal balloon of the isolation catheter to isolate a segment of the blood vessel or other body cavity;
d. providing treatment apparatus having a portion adapted to be inserted into the central lumen and having manual actuator means, the portion adapted to be inserted into the central lumen including a distal end and a lumen for passage of fluid to said distal ends, the lumen having a wall, and at least one opening through the wall of the lumen proximate the distal end;
e. introducing into the central lumen the portion of the treatment apparatus adapted to be inserted into the central lumen and advancing it to the isolation portion so that the at least one opening through the wall of the lumen is proximate the at least one opening through the wall of the central lumen; and,
f. passing fluid in the lumen so that fluid passing through the at least one opening through the wall of the lumen interacts with the isolated segment of the blood vessel or other body cavity through the at least one opening through the wall of the central lumen.

18. A method of treating unwanted material in a segment of a blood vessel or other body cavity, comprising the steps of:
a. providing an isolation catheter having a central lumen, an inflation lumen, a proximal balloon, a distal balloon, and an isolation portion between the proximal balloon and the distal balloon, the isolation portion having at least one opening through a wall of the central lumen;
b. introducing the isolation catheter into a blood vessel or other body cavity and positioning the isolation catheter at a portion of the blood vessel or other body cavity to be treated;
c. inflating the proximal balloon and the distal balloon of the isolation catheter to isolate a segment of the blood vessel or other body cavity;
d. providing a jet assembly comprising a high pressure tube having proximal and distal ends for passage of high pressure fluid between the proximal and distal ends, a fluid jet emanator in fluid communication with the high pressure tube at the distal end, and manual actuator means proximate the proximal end;
e. introducing the distal end of the high pressure tube and the fluid jet emanator into the central lumen of the isolation catheter using the manual actuator means and positioning the fluid jet emanator proximate the isolation portion using the manual actuator means;
f. providing a fluid source and a high pressure fluid pump;
g. connecting the high pressure fluid pump to the fluid source and the high pressure tube;
h. actuating the high pressure fluid pump to pump fluid from the fluid source into the high pressure tube at high pressure so that fluid emanates from the fluid jet emanator to create at least one fluid jet; and,
i. using the fluid jet(s) to treat unwanted material in the isolated segment of the blood vessel or other body cavity via fluid communication through the at least one opening through the wall of the central lumen.

19. The method of treating unwanted material of claim 18, wherein the provided jet assembly has inflow and outflow orifices and further comprising the step of using the inflow and outflow orifices to create cross stream jets which create recirculation through the at least one opening through the wall of the central lumen.

20. The method of treating unwanted material of claim 18, further comprising the steps of:
a. providing an effluent collector; and,
b. using the isolation catheter, the jet assembly, and the effluent collector to remove unwanted material from the isolated segment of the blood vessel or other body cavity.

21. The method of treating unwanted material of claim 18, further comprising the step of using the manual actuator means to move the fluid jet emanator proximate a plurality of windows in the wall of the central lumen to enhance maceration of unwanted material.

22. A method of treating a segment of a blood vessel or other body cavity, comprising the steps of:
a. providing an isolation catheter having a central lumen, an inflation lumen, a proximal balloon, a distal balloon, and an isolation portion between the proximal balloon and the distal balloon, the isolation portion having at least one opening through a wall of the central lumen;
b. introducing the isolation catheter into a blood vessel or other body cavity and positioning the isolation catheter at a portion of the blood vessel or other body cavity to be treated;
c. inflating the proximal balloon and the distal balloon of the isolation catheter to isolate a segment of the blood vessel or other body cavity;
d. providing a medication delivery device adapted to be inserted into the central lumen of the isolation catheter and having a proximal end and a distal end, a lumen for passage of medication between the proximal and distal ends, and at least one opening in the wall of the lumen proximate the distal end;
e. introducing the medication delivery device into the central lumen of the isolation catheter and positioning the medication delivery device to locate the at least one opening in the lumen of the medication delivery device proximate the at least one opening through the wall of the central lumen in the isolation portion of the isolation catheter;
f. providing a medication source and a medication pump;
g. connecting the medication pump to the medication source and to the medication delivery device;
h. actuating the medication pump to pump medication from the medication source into the medication delivery device so that medication passes from the lumen of the medication delivery device through the at least one opening and through the at least one opening through the wall of the central lumen in the isolation portion of the isolation catheter; and,
i. using the medication to treat the isolated segment of the blood vessel or other body cavity.

23. The method of claim 22, further comprising the steps of:
a. providing sealing means proximate the distal end of said central lumen to reduce leakage of fluid between said distal end of said central lumen and the distal end of the medication delivery device disposed therein; and,
b. actuating the distal seal prior to delivering medication.

24. The method of claim 22, further comprising the steps of:
a. removing the medication delivery device from the isolation catheter;
b. providing a jet assembly comprising a high pressure tube having proximal and distal ends for passage of high pressure fluid between the proximal and distal ends, a fluid jet emanator in fluid communication with the high pressure tube at the distal end, and manual actuator means proximate the proximal end;
c. introducing the distal end of the high pressure tube and the fluid jet emanator into the central lumen of the isolation catheter using the manual actuator means and positioning the fluid jet emanator proximate the isolation portion using the manual actuator means;
d. providing a fluid source and a high pressure fluid pump;
e. connecting the high pressure fluid pump to the fluid source and the high pressure tube;
f. actuating the high pressure fluid pump to pump fluid from the fluid source into the high pressure tube at high pressure so that fluid emanates from the fluid jet emanator to create at least one fluid jet; and,
g. using the fluid jet(s) to treat unwanted material in the isolated segment of the blood vessel or other body cavity via fluid communication through the at least one opening through the wall of the central lumen.

25. The method of claim 24, further comprising the steps of:
a. providing an effluent collector; and,
b. using the effluent collector to receive fluid from the central lumen and thereby remove unwanted material.

26. A method of treating a segment of a blood vessel or other body cavity, comprising the steps of:
a. providing an isolation catheter having a central lumen, an inflation lumen, a proximal balloon, a distal balloon, and an isolation portion between the proximal balloon and the distal balloon, the isolation portion having at least one opening through a wall of the central lumen;
b. introducing the isolation catheter into a blood vessel or other body cavity and positioning the isolation catheter at a portion of the blood vessel or other body cavity to be treated;
c. inflating the proximal balloon and the distal balloon of the isolation catheter to isolate a segment of the blood vessel or other body cavity;
d. providing a plurality of treatment apparatuses each adapted to be advanced into the central lumen of the isolation catheter; and,
e. introducing into the central lumen of the isolation catheter each of the provided treatment apparatuses one at a time, operating adjacent to the at least one opening in the isolation portion each of the introduced treatment apparatuses one at a time, and removing each of the introduced treatment apparatuses one at a time.

27. A method of treating unwanted material in a blood vessel or other body cavity, comprising the steps of:
a. providing an isolation catheter having a central lumen, an inflation lumen, a proximal balloon, a distal balloon, and an isolation portion between the proximal balloon and the distal balloon, the isolation portion having at least one opening through a wall of the central lumen;
b. introducing the isolation catheter into a blood vessel or other body cavity and positioning the isolation catheter at a portion of the blood vessel or other body cavity to be treated;
c. inflating the proximal balloon and the distal balloon of the isolation catheter to isolate a portion of the blood vessel or other body cavity;
d. providing a jet assembly comprising a high pressure tube having proximal and distal ends for passage of high pressure fluid between the proximal and distal ends, a fluid jet emanator in fluid communication with the high pressure tube at the distal end, and manual actuator means proximate the proximal end;
e. introducing the distal end of the high pressure tube and the fluid jet emanator into the central lumen of the isolation catheter using the manual actuator means and positioning the fluid jet emanator past the distal end of the central lumen of the isolation catheter and beyond the isolated portion of the blood vessel or other body cavity;
f. providing a fluid source and a high pressure fluid pump;
g. connecting the high pressure fluid pump to the fluid source and the high pressure tube;
h. actuating the high pressure fluid pump to pump fluid from the fluid source into the high pressure tube at high pressure so that fluid emanates from the fluid jet emanator to create at least one fluid jet; and,
i. using the fluid jet(s) to treat unwanted material in the blood vessel or other body cavity while the isolation balloons are isolating a portion of the blood vessel or other body cavity.

28. The method of treating unwanted material of claim 27, wherein the jet assembly comprises inflow and outflow orifices and further comprising the steps of:
a. providing an effluent collector;
b. using the inflow and outflow orifices to create cross stream jets to enhance break up of unwanted material in the blood vessel or other body cavity; and,
c. using the isolation catheter, the jet assembly, and the effluent collector to remove unwanted material from beyond the isolated portion of the blood vessel or other body cavity.

29. An isolation thrombectomy catheter system comprising:
a. an isolation catheter having openings in an isolated portion;
b. a jet assembly including a fluid jet emanator and a manual actuator;
c. a fluid source;
d. a high pressure pump; and,
e. an effluent collector.
